(19) United States
(12) Patent Application Publication
(10) Pub. No.: US 2009/0048654 A1
(43) Pub. Date: Feb. 19, 2009

(54) Deployment System for Soft Stents

(75) Inventors: Kevin Chmura, Winston-Salem, NC (US); John M. Curran, Winston-Salem, NC (US); Richard W. Ducharme, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/190,737

(22) Filed: Aug. 13, 2008

Related U.S. Application Data
(60) Provisional application No. 60/955,940, filed on Aug. 15, 2007.

(51) Int. Cl. A61F 2/84

(52) U.S. Cl. 623/1.11

(57) ABSTRACT

A stent delivery system and a method for delivering a stent are provided. The stent delivery system includes an outer sheath having a proximal portion, a distal portion and a first lumen extending at least partially through the sheath. The system further includes an inner shaft slidably received within the first lumen and extending at least partially through the sheath. A tubular non-expandable stent is slidably positionable within the first lumen, disposed distal to the inner shaft and operably contacts a pushing surface on the inner shaft. The inner shaft and the stent are slidable relative to the outer sheath, the outer sheath providing sufficient rigidity to the stent for delivery of the stent to a delivery site.

### RELATED APPLICATIONS

This application claims the benefit of U.S. Provisional Application No. 60/955,940, filed Aug. 15, 2007, which is incorporated by reference herein in its entirety.

### TECHNICAL FIELD

This invention generally relates to medical devices, and more particularly to devices for delivering an implantable prosthesis to a target anatomy.

### BACKGROUND

Prosthetic devices may be placed in vessels and ducts for a number of medical procedures. Typically, placement of the prosthetic devices into the vessels and ducts functions to maintain an open passage through the vessel or duct. For example, where a bilary or pancreatic duct becomes occluded, it is often desirable to facilitate drainage through the duct by the placement of a tubular prosthesis within the occluded area. In some procedures, stents have been used to maintain an open passage. Flexibility of the stent is important to avoid irritation of the placement site with a rigid stent. For example, patients may develop pancreatitis and morphological changes or strictures due to irritation at the implant site by a stent that is too stiff.

Placement of a stent within a patient can be problematic due to the patient anatomy and stent flexibility. For example, placement of the stent in the biliary tree can be difficult, since a deployment system must make a severe turn from the duodenum through the opening of the common bile duct. The geometry of cancerous biliary or pancreatic ducts is also very tortuous. In addition, the narrow passageways of the biliary and pancreatic ductal system or the urinary system restrict the diameter of the delivery system that may be used for delivering a stent within the narrow passageways. Similarly, small diameter flexible stents suitable for biliary and pancreatic ducts or the urinary system may be problematic to deliver due to the size of the stent and the flexibility of the stent. For example, buckling or kinking of the stent during delivery to the target site may occur in stents that are flexible and soft enough such that these stents may be longitudinally compressible during delivery.

In some delivery systems, the stent is delivered to the implantation site using a catheter. For example, the biliary or pancreatic stent may be mounted on a guiding catheter that is fed over a wire guide into the biliary tree. To deploy the stent from over the guiding catheter, a pushing catheter is used to contact a proximal end of the stent and urge the stent forward over the guiding catheter until deployment occurs and the stent is released at the implantation site. Stents may also be delivered by placing a stent directly over a wire guide and pushing the stent with a pushing catheter. Typically, stents with smaller French sizes (generally about 7 FR and smaller, limited by the diameter of the wire guide) are delivered by direct placement of the stent on the wire guide. When the stent is relatively stiff, the stent may be delivered to a site without buckling. However, these types of deliverable stents do not address the problem of irritation within the duct due to the presence of the stiff stent. When a soft stent that may be longitudinally compressible during delivery is deployed using a pushing catheter to push on the end of the stent as the stent advances over the guiding catheter, the stent may buckle or accordion during delivery. Buckling of the stent may make delivery to the implantation site difficult or impossible if the stent cannot advance past the stricture into position. In addition, buckling of the stent may interfere with proper positioning of the stent or irritate the passageway of the biliary tree or urinary system as the stent is being delivered. If the stent buckles during delivery, the buckling may cause inadvertent displacement of the stent relative to the pushing catheter and affect proper placement of the stent in the stricture. In addition, with some materials, kinking of the stent may damage the stent and render the stent unusable.

What is needed is a stent introducer system that enables deployment of a longitudinally compressible stent to the delivery site without buckling the stent during delivery.

### SUMMARY OF THE INVENTION

Accordingly, it is an object of the present invention to provide a stent delivery system and method having features that resolve or improve on one or more of the above-described drawbacks.

The foregoing object is obtained by providing a stent delivery system having an outer sheath having a proximal portion, a distal portion and a first lumen extending at least partially through the sheath. The system further includes an inner shaft slidably received within the first lumen and extending at least partially through the sheath. A tubular stent is slidably positionable within the first lumen and at least a portion of the stent operably contacts a pushing surface on the inner shaft. The inner shaft and the stent are slidable relative to the outer sheath and the outer sheath provides sufficient rigidity to the stent for delivery of the stent to a delivery site.

In another aspect, a method of delivering a pancreatic stent using a delivery system of the present invention. The method includes providing a stent delivery system, advancing the delivery system to a delivery site, deploying the stent into the delivery site by sliding the shaft and the stent relative to the sheath, the sheath providing sufficient rigidity to the stent for delivery of the stent to the delivery site and withdrawing the shaft and the sheath.

### BRIEF DESCRIPTION OF THE DRAWINGS

FIG. 1 is a partial, cross-sectional view of the stent delivery apparatus of the present invention;

FIG. 2A is a cross-sectional view of a distal portion of the delivery apparatus shown in FIG. 1 along line 2-2;

FIG. 2B is a cross-sectional view of an alternative embodiment shown in FIG. 2A;

FIG. 3 is a partial, cross-sectional view of an alternative embodiment of the distal portion of the stent delivery apparatus shown in FIG. 1;

FIG. 4 is a side view of a stent suitable for delivery using the present invention;

FIG. 5 is a side view of an alternative stent suitable for delivery using the present invention;

FIG. 6 is a side view of an alternative stent suitable for delivery using the present invention;

FIG. 7A depicts a method of delivering the stent without an outer sheath within the common bile duct;

FIG. 7B illustrates buckling of the stent at the common bile duct with out the outer sheath;

FIGS. 8A-8B depict a method of delivering the stent using the system embodied in FIG. 1; and

FIG. 8C depicts an alternative method of delivering the stent within the common bile duct using the system embodied in FIG. 1.

### DETAILED DESCRIPTION

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician using the deployment system. Hence the term “distal” means the portion of the deployment system which is farthest from the physician and the term “proximal” means the portion of the deployment system which is nearest to the physician.

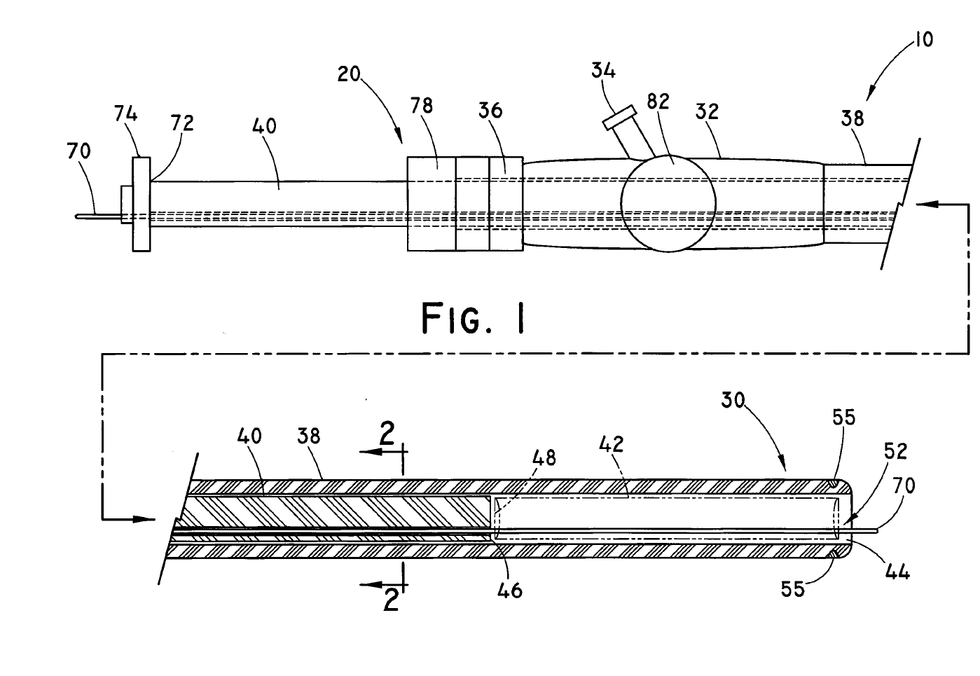

FIG. 1 illustrates a stent introducer apparatus 10 in accordance with an embodiment of the present invention. The stent introducer apparatus 10 includes a proximal portion 20 and a distal portion 30. The proximal portion 20 includes a handle 32 that may include an injection port 34 and a hub 36. An outer sheath 38 is operably connected to the handle 32 and extends distally from the handle 32. The stent apparatus 10 further includes an inner shaft 40 for advancing a stent 42 for deployment within a duct or vessel. The inner shaft 40 is slidably received within a lumen 44 defined through at least a portion of the outer sheath 38 and is proximal to the stent 42 within the lumen 44. The outer sheath 38 may include a Touhy-Borst adapter to keep the relative position between the outer sheath 38 and the inner shaft 40 fixed until the stent 42 is ready to be deployed. Any type of mechanical interlock may also be used. The shaft 40 includes a distal end 46 for contacting a proximal end portion 48 of the stent 42.

The stent 42 is slidably received in a distal end portion 52 of the lumen 44 of the outer sheath 38 and is disposed distally of the shaft 40. The stent 42 may be any kind of tubular, non-expandable stent known in the art that is suitable for placement in a passageway of a patient. By way of non-limiting example, the stent 42 may be a pancreatic, biliary or urological stent. The stent 42 may be longitudinally compressible during delivery due to the geometry of the stent 42 and the materials forming the stent, such that the stent 42 is not consistently adequately independently pushable absent the outer sheath 38. The outer sheath 38 provides protection from deformation of the stent 42 during delivery of the stent 42 to the delivery site. The sheath 38 provides sufficient rigidity to the stent 42 to allow the stent 42 to be pushable. The sheath 38 may be configured to prevent frictional engagement of the stent 42 with the tissue as the stent is navigated through the bodily passageways and to avoid inwardly directed lateral pressure The sheath 38 may, but is not required to provide compression for the stent 42, for example when the stent 42 includes retention members (discussed below).

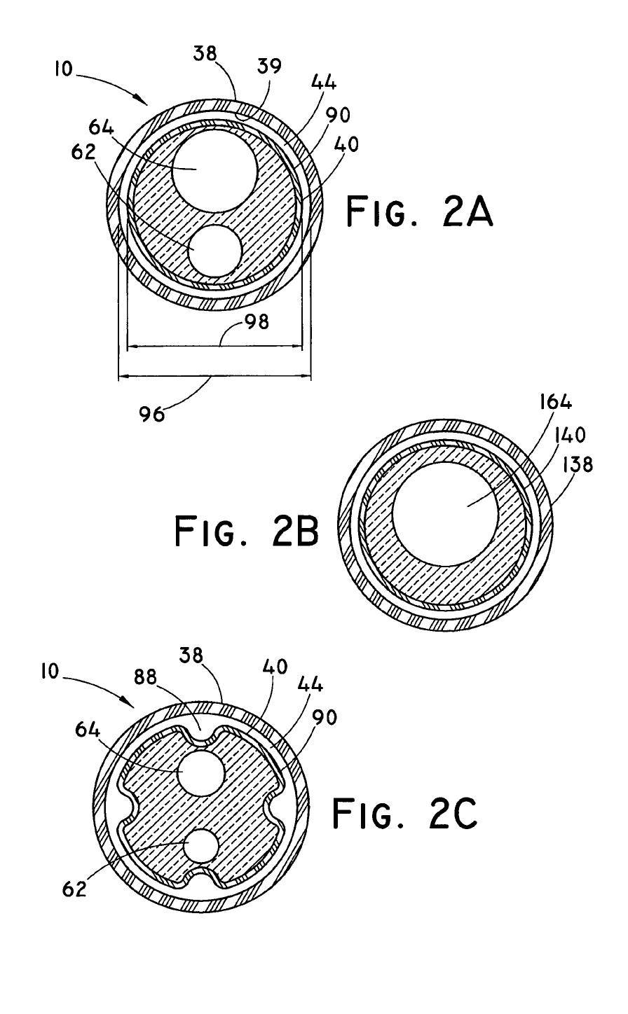

The inner shaft 40 may further include one or more lumens 62, 64 defined through a portion of the shaft 40. FIG. 2A illustrates a cross-sectional view through the apparatus 10 along the line 2-2 shown in FIG. 1. As shown, the lumens 62, 64 may be off-set from a central longitudinal axis 66 of the apparatus 10 and the lumens 62, 64 may be of unequal size. The apparatus 10 may include one, two, three or more lumens defined at least partially through the shaft 40. Each lumen may be sized and shaped according to the purpose of the lumen. For example, one lumen 64 may be configured to receive a wire guide 70. Another lumen 62 may be configured for connection to the port 34 for flushing the lumen 44 of the outer sheath 38 and/or for providing fluid delivery during deployment of the stent 42. Alternatively, a single lumen 164 may extend at least partially through the shaft 140, as shown in FIG. 2B. For example, the single lumen 164 may be sized to receive the wire guide 70 for use with the stent 42 that is sized just slightly larger than the wire guide 70 for delivery to a narrow passage within the body. The shaft 140 may be received in the outer sheath 138.

As shown in FIG. 2C, the shaft 40 may include one or more grooves 88 on an exterior surface 90. The grooves 88 may extend the length of the shaft 40 or at least partially along a portion of the shaft 40. The grooves 88 may be provided to facilitate slidable movement of the shaft 40 with respect to the sheath 38 by providing a fluid path between the shaft 40 and the sheath 38. In some embodiments, the shaft 40 may include a metal portion at a proximal portion to facilitate retraction of the sheath 38 during deployment. Additional materials and features may also be used for the proximal portion of the shaft 40 to facilitate retraction of the sheath 38 as will be understood by one skilled in the art.

In some embodiments, a shaft proximal end 72 includes a luer-lock fitting 74 for releasably fixing the wire guide 70 relative to shaft 40 as shown in FIG. 1. The handle 32 may further include a releasable locking mechanism 78 at the hub 36 for releasably locking the shaft 40 to the outer sheath 38. The handle 32 may also include a gripping member 82.

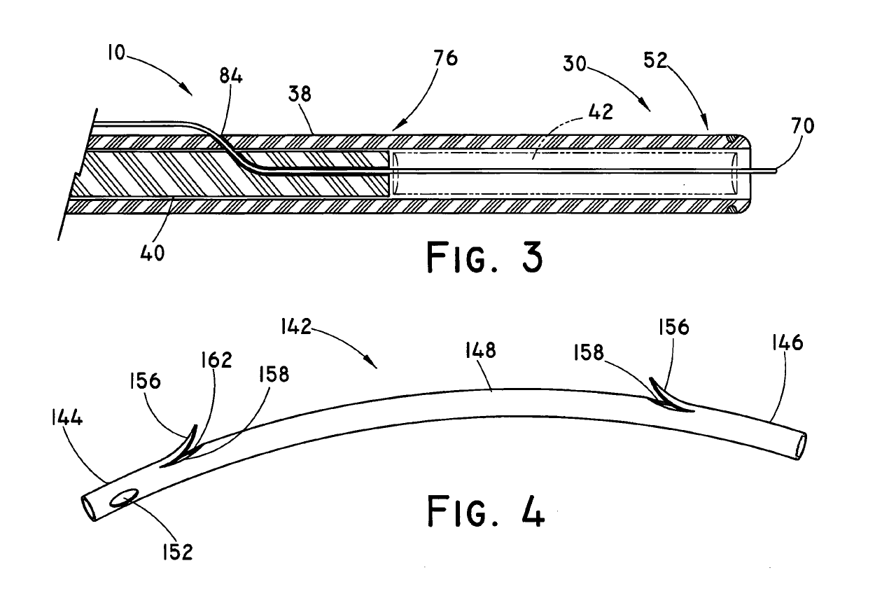

In an alternate embodiment shown in FIG. 3, the wire guide 70 extends through a distal portion 76 of the shaft 40 and exits through an aperture 84 positioned along the length of outer sheath 38. In this embodiment, the wire guide 70 extends through the distal end portion 52 of the outer sheath 38, through the portion 76 of the shaft 40 and passes through stent 42 before exiting the stent introducer apparatus 10. For example, the wire guide 70 may extend through the distal end portion 52 of the sheath 38 for a distance of about 20 cm. Any number of apertures 84 positioned at any location along the length of the apparatus 10 is contemplated. The aperture 84 provides the stent introducer apparatus 10 of the present invention with the capability to quickly change devices. In particular, by extending the wire guide 70 through only a distal portion of the sheath 38, the introducer apparatus 10 can be removed from the wire guide 70 having a length substantially shorter than the length necessary if the wire guide 70 were extended through the entire length of the wire guide lumen 64 in the shaft 40.

The outer sheath 38 may be made from a material that allows the sheath to be sufficiently flexible yet having adequate columnar strength to navigate the patient's ductal system. In some embodiments, the outer sheath is made primarily of a substantially clear polymer such as polytetrafluorothylene (PTFE). Additional possible materials include, but are not limited to the following, polyethylene ether ketone (PEEK), fluorinated ethylene propylene (FEP), perfluoroalkoxy polymer resin (PFA), polyamide, polyurethane, high density or low density polyethylene, and nylon including multi-layer or single layer structures and may also include reinforcement wires, braid wires, coils, coil springs and or filaments. In some embodiments, the outer sheath 38 is formed from a lubricious material such as PTFE and the like for easy slidability of the inner shaft 40 and the stent 42 within the outer shaft 38. An inner surface 39 of the outer sheath 38 may also be treated with materials to make the inner surface 39 more lubricious. The outer sheath 38 may also be coated or impregnated with other compounds and materials to achieve the desired properties. Exemplary coatings or additives include, but are not limited to, parylene, glass fillers, silicone hydrogel polymers and hydrophilic coatings. In some embodiments, the thickness of the outer sheath wall may range from about 0.005-0.030 inch.

The size of the outer sheath 38 will depend on the size of the inner shaft 40 and the stent 42. In some embodiments, the sheath 38 will be sized to tightly slidably receive the shaft 40 and the stent 42. For example, the sheath 38 may have an outer diameter 96 that is about 1-3 French (Fr) greater than the stent 42 outer diameter. The shaft 40 may have an outer diameter 98 that is just slightly larger than the stent 42 outer diameter. The relationship between the sheath outer diameter 96 and the shaft 40 outer diameter 98 is shown in FIG. 2A. The length of the sheath 38 and the shaft 40 may be about 150-300 cm. The stent 42 for delivery using the sheath 38 and shaft 40 may have an outer diameter of about 3-10 Fr, preferably about 3-7 Fr, and have a length of about 4-22 cm. Other sizes and lengths for the sheath 38, shaft 40, and stent 42 are possible for use with the present invention. These sizes and lengths have been provided for illustrative purposes.

The shaft 40 may be made from a material that allows the shaft to be sufficiently flexible yet have adequate columnar strength and be slidable within the sheath 38. Possible materials include, but are not limited to PTFE, PEEK, polyethylene, nylon, polyimide, and polyurethane. The shaft 40 may be sized and shaped such that the outer diameter of the shaft is dimensioned to take up most of the inner diameter of the sheath 38. The outer diameter of the distal end 46 of the shaft 40 generally depends on the type of the stent 42 to be delivered and the inner diameter of the outer sheath 38. The shaft 40 may also be coated or formed from materials having a lubricious surface, such as PTFE, nylon, FEP, PEEK, polyethylene and the like.

The wire guide 70 may be any type of wire guide known in the art suitable for entering tortuous passageways in the body. The wire guide 70 should be sized and shaped to fit and extend at least partially through the lumen 64 in the shaft 40. In some embodiments, the wire guide 70 may be about 0.018 to about 0.035 inch in diameter (coated or uncoated) and about 205 cm in length for a device that allows exchange at the distal end portion of the shaft and up to about 1000 cm in length. In some embodiments, the wire guide 70 may be about 480 cm or about 660 cm in length. Other diameters and lengths may be used as these sizes are presented only for illustrative purposes.

As discussed above, the stent 42 may be any stent suitable for deployment into a bodily passageway. In some embodiments, the stent may have an outer diameter of about 3-5 Fr, although larger stents may be used, for example, about 5-7 Fr, about 7-9 Fr and the like. If smaller stents become available, i.e. smaller than 3 Fr, the apparatus 10 would be suitable for delivering the smaller stents without buckling during delivery. Similarly, any soft stent may be delivered using the apparatus 10 described herein where the stent is not placed over a guiding catheter or a pushing catheter for delivery. The stent may be made from materials so that the stent is soft enough to eliminate or reduce irritation at the implantation site that occurs with a rigid stent, thus reducing the risk of pancreatitis or other ductal changes in the biliary and urological ducts. These soft stents tend to buckle without the outer sheath of the present invention overlying the stent for delivering the stent to the implant site. Suitable materials for the stent for use with the delivery system of the present invention include, but are not limited to the following, SOF-FLEX®, a type of polyether urethane, silicone, block polymers, urethane, polyethylene, PTFE and combinations thereof.

By way of non-limiting example, the stent may be provided for facilitating the drainage of fluids within an obstructed duct. As shown in FIGS. 4-6, a tubular drainage stents for implantation into an obstructed duct or bodily passage, such as the bile duct, pancreatic duct, urethra, etc., is provided. The stents are generally tubular, non-expandable stents that include a solid wall over a majority of the length of the stent. Small holes or flaps may be included, as described below. A stent 142, shown in FIG. 4, includes a first end 144 for drainage into a duct, vessel, organ, etc., and a second end 146 that receives the fluid or other material. An elongate tubular region 148 extends between the ends 144, 146. The elongate tubular region may be closed or alternatively, may include one or more openings 152 to facilitate fluid flow. The tubular stent 142 is typically non-expanding, unlike the wire or open-frame stents known in the art. The stent 142 is commonly placed either to establish or maintain patency of the bodily passage or to drain an organ or fluid source, such as the gall bladder or urinary bladder.

The tubular drainage stent 142 may also include a retention members 154, 156 at one or more end portions 144, 146 such as flaps, pigtail loops, etc. The number, size and orientation of the retention numbers that may optionally be included may be modified to accommodate the migration-preventing requirements of the particular stent to be implanted. The retention members may be included near one end portion 144 or 146 or both end portions 144, 146 of the tubular stent 142. In some embodiments, the retention members may be formed by slicing small longitudinal sections 158 in the stent 142 and orienting the sliced sections 158 radially. The sliced sections forming the retention members 154, 156 shown in FIG. 4 may be formed such that the slices 158 do not form holes within the tubular stent 142, for example at the retention member 156. Alternatively, the slices 158 may be provided such that a small hole 162 is formed in the stent 142 where the retention member 154 is formed. Any number of retention members may be included and may be arranged in rows around one or both end portions 144, 146.

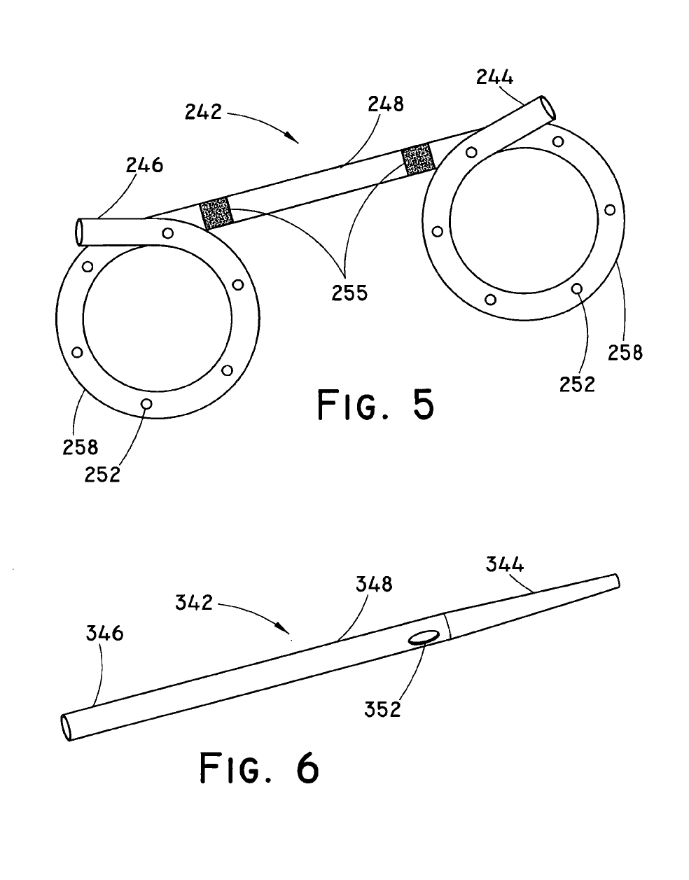

As shown in FIG. 5, another type of exemplary tubular stent is illustrated. Stent 242 includes a first end 244 for drainage into a duct, vessel, organ, etc., and a second end 246 that receives the fluid or other material. A tubular region 248 including a solid wall over a majority of its length extends between the ends 244, 246 similar to the stent 142 described above. The stent 242 further includes retention members 258 in the form of pigtail loops for preventing migration of the stent 242. The retention members 258 may include openings 252 to facilitate drainage.

As shown in FIG. 6, a simple tubular stent 342 is illustrated. By way of example, the stent 342 may be used in pancreatic, biliary or urological ducts. The stent 342 includes a first end portion 344 and a second end portion 346. A tubular region 348 extends between the end portions 344, 346 and may optionally include an opening 352 formed in the tubular portion 348. One or both end portions 344, 346 may be tapered.

Suitably shaped tubular stents known in the art include, but are not limited to, a ST-2 SOEHENDRA TANNENBAUM® stent, a COTTON-LEUNG® stent, a COTTON-HUIBREGTSE® stent, a GEENEN® Pancreatic Stent, a JOHLIN® Pancreatic Wedge Stent, or a ZIMMON® Pancreatic (available from Cook Endoscopy, Inc., Winston-Salem, N.C.). Other tubular stents known in the art are also suitably shaped for delivery using the stent introducer apparatus of the present invention. The stent of the present invention may be similarly shaped, but is also formed from a material and is of a size that the stent is longitudinally compressible and may not be independently pushable absent the outer sheath of the delivery system. For example, the stent of the present invention may be formed from a material such as polyether urethane having a lower gurley stiffness, lower durometer and lower modulus than a stent formed from a material such as polyethylene. In some embodiments, the stent for use with the delivery system of the present invention may have a resistance to bending less than about 1,300,000 mgs/in². Typically, polyethylene stents known in the art are stiffer and have a higher resistance to bending that is greater than about 1,300,000 mgs/in² and may be less than about 2,3321,000 mgs/in².

The stent for delivery using the apparatus 10 may be made from any suitable material that is biocompatible and flexible enough to be longitudinally compressible for positioning in a bodily passage to allow fluid flow therethrough. The stent may be made from plastic materials known in the art. The stent materials may be substantially non-biodegradable or biodegradable.

Radiopaque markers may be provided on the distal portion 52 of the sheath 38, the distal end 46 of the shaft 40 and/or the stent 42. Alternatively, portions of the stent introducer apparatus 10 may be made from materials that are radiopaque themselves. Exemplary radiopaque bands 55, 255 are shown on the sheath 38 and the sent 242 in FIGS. 1B and 5, respectively.

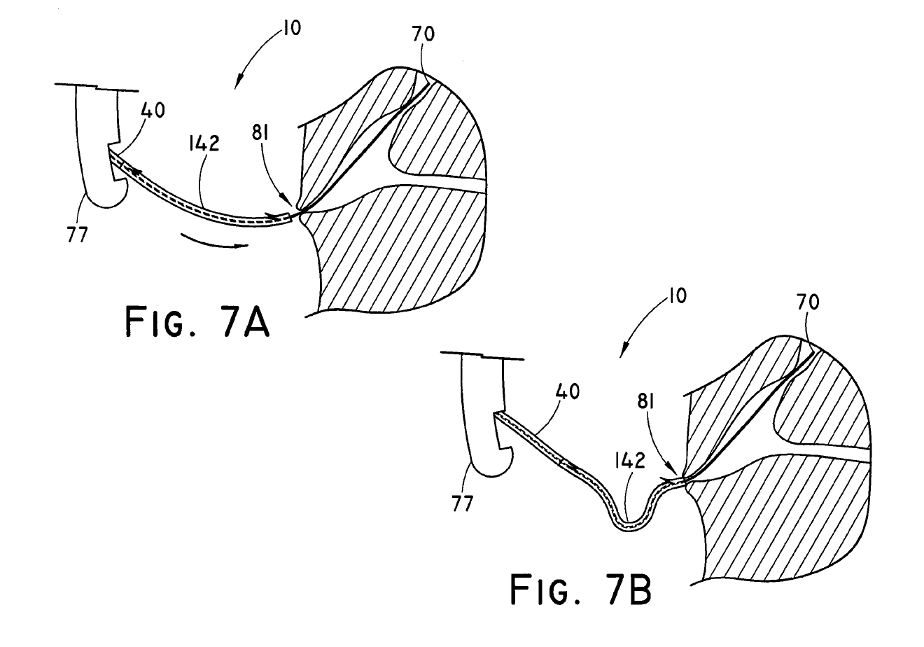

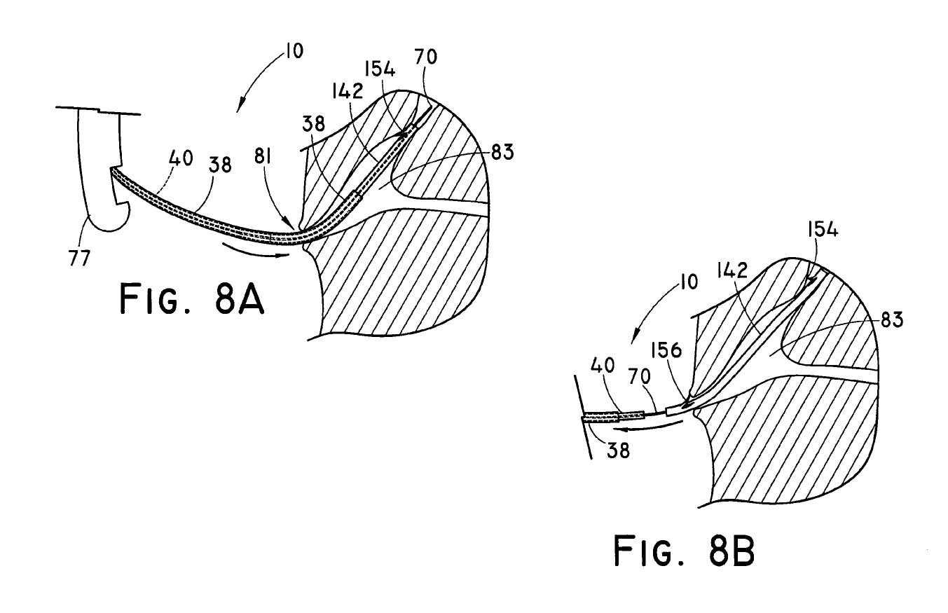

In operation, the stent introducer apparatus 10 may be used to place the stent in the bodily lumen. FIGS. 7A and 7B illustrate buckling of the stent during delivery when the soft stent is delivered without an outer sheath. As shown in FIGS. 7A and 7B, the wire guide 70 enters a duct 81 and the shaft 40 urges the stent 142 to the duct. As shown in FIG. 7B, the stent 142 buckles against the duct 81 and is not properly positionable.

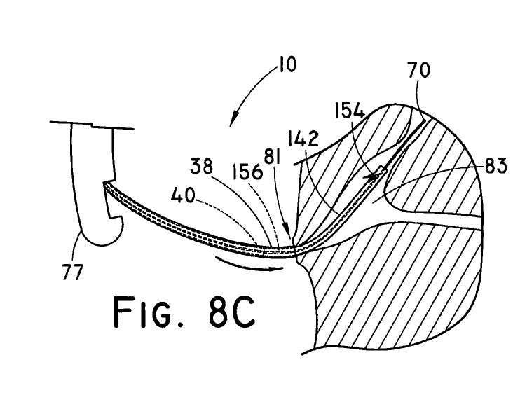

FIGS. 8A-8C illustrate the stent delivery system of the present invention having the outer sheath 38 to facilitate delivery of the stent 142 that is not adequately and consistently independently pushable to the implantation site through the duct 81. The stent is positioned within the sheath 38 for passage thought the body lumens until the implantation site is reached. By covering the stent with the sheath 38 during delivery, sufficient rigidity is provided for the stent by the sheath so that kinking or accordioning of the stent is avoided as the stent passes through the tortuous pathway to the implantation site. For illustrative purposes, the stent 142 will be used for delivery to the bodily lumen, however, any stent may be similarly delivered.

As shown in FIG. 8A, the sheath 38 is advanced over the wire guide 70 out of an endoscope 77 through an ampullary orifice 81 and a duct 83. The sheath 38 provides rigidity to the stent 142 as the stent 142 moves toward the delivery site within the duct 83. The sheath 38 provides a bridge across the duct 83 for positioning of the stent 142. The shaft 40 urges the stent 142 into position in the duct 83 by pushing the stent 142 out of the sheath 38. The sheath may be withdrawn at the same time so that the stent 142 is positioned within the duct 83. The sheath 38 may provide a bridge across the duct 83 so that the stent 142 does not compress longitudinally as the stent 142 is being delivered into the duct 83. As the stent 142 exits the sheath 38, the retention member 154 on the stent 142 expands outwardly to contact the duct wall and hold the stent 142 in position. Once the stent 142 is in the proper position for deployment, as depicted in FIG. 8B, the stent introducer apparatus 10 is withdrawn and the stent 142 is positioned in the duct 83 with the retention members 154, 156 extended outwardly against the tissue. Subsequent deployments of additional stents can be also be made using the same technique over the original wire guide.

As shown in FIG. 8C, the sheath 38 may be used to position the stent 142 at the orifice 81 so that the stent is positioned in the duct 81 without extending the sheath 38 through the duct 83, for example, where the stricture is too narrow to permit the sheath 38 extend through the duct 83. The sheath 38 is advanced over the wire guide 70 out of the endoscope 77 to the ampullary orifice 81 but not through the duct 83. The wire guide 70 extends into the duct 83. The shaft 40 urges the stent 142 into position in the duct 83 by pushing the stent 142 out of the sheath 38 and through the orifice 81. As the stent 142 exits the sheath 38 and enters the duct 83, the retention member 154 on the stent 142 expands outwardly to contact the duct wall and hold the stent 142 in position. The shaft 40 pushes the stent 142 out of the sheath 38 until the stent 142 is properly positioned in the duct 83. Once the sheath 38 is fully withdrawn from the stent 142, both the retention members 154, 156 may expand outwardly to hold the stent 142 in position.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims. For example, the invention has been described in the context of the biliary system for illustrative purposes only. Application of the principles of the invention to any other bifurcated lumens or vessels within the body of a patient, including areas within the digestive tract such as the pancreatic system, as well as areas outside the digestive tract such as other vascular systems, by way of non-limiting examples, are within the ordinary skill in the art and are intended to be encompassed within the scope of the attached claims.

1. A stent delivery system comprising;
an outer sheath comprising a proximal portion, a distal portion and a first lumen extending at least partially through the sheath;
an inner shaft slidably received within the first lumen, the inner shaft comprising a distal end having a pushing surface extending across at least a portion of the distal end; and
a tubular, non-expandable stent having a longitudinally compressible solid wall over a majority of its length, and slidably positionable within the first lumen, the stent being disposed distally of the inner shaft and at least a portion of the stent operably contacting the pushing surface,
wherein the inner shaft and the stent are slidable relative to the outer sheath, the outer sheath providing sufficient rigidity to the stent for delivery of the stent to a delivery site.

2. The stent delivery system of claim 1, wherein an outer diameter of the stent is between about 3 French to about 7 French.

3. The stent delivery system of claim 1, wherein an outer diameter of the stent is about 5 French or less.

4. The stent delivery system of claim 1, wherein the stent comprises a material selected from the group consisting of plastics, silicone, urethane, polyethylene, PTFE, FEP and combinations thereof.

5. The stent delivery system of claim 1, wherein the stent comprises polyether urethane.

6. The stent delivery system of claim 1, wherein the stent has a resistance to bending less than about 1,300,000 mgs/in².

7. The stent delivery system of claim 1, wherein the shaft comprises a wire guide lumen extending at least partially through the shaft.

8. The stent delivery system of claim 7, wherein the system further comprises a wire guide slidably received with in at least a portion of the wire guide lumen.

9. The stent delivery system of claim 1, further comprising a handle operably connected to the outer sheath, the handle comprising a flushing port.

10. The stent delivery system of claim 1, wherein the stent further comprises at least one retention member for substantially preventing migration of the stent.

11. The stent delivery system of claim 10, wherein the at least one retention member comprises a radially extending flap formed by slicing a small longitudinal section in the stent.

12. The stent delivery system of claim 10, wherein the at least one retention member comprises a pigtail loop.

13. The stent delivery system of claim 1, wherein the inner shaft is configured for pushing the stent into the delivery site without using a guiding catheter.

14. The stent delivery system of claim 1, wherein the outer sheath comprises PTFE.

15. The stent delivery system of claim 1, wherein the inner shaft further comprises grooves in an outer surface of the shaft.

16. A method for delivering a pancreatic stent comprising;
providing a stent delivery system comprising:
an outer sheath comprising a first lumen extending at least partially through the sheath;
an inner shaft slidably received within the first lumen, the inner shaft comprising a distal end having a pushing surface extending across at least a portion of the distal end; and
a tubular non-expandable stent slidably positionable within the first lumen, disposed distal to the inner shaft and at least a portion of the stent operably contacting the pushing surface;
advancing the delivery system to a delivery site;
deploying the stent into the delivery site by sliding the shaft and the stent relative to the sheath, the sheath providing sufficient rigidity to the stent for delivery to the site; and
withdrawing the shaft and the sheath.

17. The method of claim 16, further comprising visualizing radiopaque markers provided on the delivery apparatus for positioning the stent at the delivery site.

18. The method of claim 16, wherein deploying the stent comprises advancing the stent into the delivery site by pushing the stent using the pushing surface of the shaft.

19. The method of claim 16, wherein deploying the stent comprises withdrawing the sheath from the delivery site while contacting the stent with the pushing surface of the shaft to hold the stent at the delivery site.

20. The method of claim 16, further comprising withdrawing the shaft into the sheath.
